(19) United States
(12) Patent Application Publication
(10) Pub. No.: US 2010/0329982 A1
(43) Pub. Date: Dec. 30, 2010

(54) PARTICLES WITH INDUCIBLE CHANGE OF SHAPE

(75) Inventors: Andreas LENDLEIN, Berlin (DE); Christian Wischke, Teltow (DE); Axel Thomas Neffe, Berlin (DE)

Correspondence Address: SUGHRUE MION, PLLC, 2100 PENNSYLVANIA AVENUE, N.W., SUITE 800, WASHINGTON, DC, 20037, US

(73) Assignee: GKSS-Forschungszentrum Geesthacht GmbH, Geesthacht (DE)

(21) Appl. No.: 12/822,857

(22) Filed: Jun. 24, 2010

(30) Foreign Application Priority Data
Jun. 24, 2009 (DE) 10 2009 027 151.1

(51) Int. Cl. A61K 49/00; A61K 9/48; A61K 47/14; A61K 39/395; A61K 39/00; C12Q 1/68; G01N 33/53; C12Q 1/02; A61K 38/14; A61K 38/17; A61K 48/00; A61K 31/715; A61K 31/56; A61K 47/30; A61K 47/32

(52) U.S. Cl. 424/9.1; 424/452; 424/501; 424/502; 424/145.1; 424/184.1; 435/6; 435/7.1; 435/29; 514/1.1; 514/7.4; 514/20.9; 514/21.2; 514/44 R; 514/54; 514/180; 514/772.3; 514/785; 514/788.1

(57) ABSTRACT

The invention relates to particles which exhibit a stimulable shape change and allow control of their uptake in active cells. The particles can be used as carrier systems for bioactive molecules or as diagnostic agents, and the spherical shape thereof has a size permitting uptake in a cell. Each particle assumes a temporary, non-spherical and thus non-uptakeable or only slightly uptakeable shape which can be transformed into a spherical and thus uptakeable shape by a suitable stimulus.

The invention relates to particles which exhibit an inducible shape change and allow active control of their uptake in cells. Said particles can be used as carrier systems for bioactive molecules or as diagnostic agents.

### RELATED ART

In many medical fields of application there is a constant need to control the release of active substances both temporally and locally, for instance as part of a drug therapy in humans and animals. Furthermore, there is a need to locally concentrate diagnostic agents in humans and animals at defined sites or in defined tissues of the organism, e.g. in imaging methods. Frequently, biological processes proceeding with participation of the immune system are of particular importance in physiology and pathology.

For immunological processing of, for example, foreign particles such as bacteria or other particles, initial uptake of active particles in cells, particularly antigen-presenting cells, is required. In therapeutic or diagnostic fields of application it would be desirable to control the timing of the cellular uptake of particles. Consequently, there is a need for solutions allowing switchable biological recognition and cellular uptake of particles.

As is well-known, the uptake of dimensionally stable particles of phagocytically active cells either is possible or uptake thereof is impossible or strongly reduced, which depends on the shape of the particles (Proc. Natl. Acad. Sci. 103 (2006) pp. 4930-4934; Pharm. Res. 25 (2008) pp. 1815-1821). Thus, investigations relating to the influence of particle geometry on recognition by active cells have revealed that particular geometries, e.g. worm-like particles, reduce the uptake rate compared to spherical particles.

### SUMMARY OF THE INVENTION

One or more of the above-mentioned problems are solved or at least mitigated with the aid of the particles according to the invention. Particles capable of undergoing an inducible change in shape are provided. A particle suitable for cellular uptake assumes a temporary, non-spherical shape prior to use, which therefore cannot be taken up or only to a minor extent, said shape being transformable into a spherical shape, i.e. uptakeable in cells, by a suitable stimulus. Accordingly, each particle assumes a temporary, non-spherical shape which therefore cannot be taken up by active cells or only to a minor extent, said shape being transformable into a spherical and thus uptakeable shape by a suitable stimulus.

The invention is based on the understanding of providing particles initially in a temporarily programmed shape unfavorable for cellular uptake. Induced by a stimulus after administering the particles, the particles assume a spherical contour allowing increased cellular uptake compared to the initial shape. In other words, it is only after re-obtaining the permanent spherical shape that the uptake of the particle by active cells is increased. In the event of particles including bioactive molecules, the desired effects caused by the bioactive molecule(s) can subsequently develop inside the cells. Such technical solutions allowing switchable biological recognition of particles for cellular uptake as a result of a specific change of particle shape have hitherto been unknown. Accordingly, a portion of the particle or the particle as a whole, apart from active substance optionally incorporated and optional further adjuvants, consists of a material which is fixed in a temporary shape and can be put to use in this shape. Using a suitable stimulus, the material and thus the particle as a whole undergoes recovery into the programmed spherical shape, i.e. from the temporary, non-uptakeable shape into the shape that can be taken up by active cells.

The term “particle” in the meaning of the present application relates to particles of a wide variety of structures. More specifically, the particle—as a whole or in portions—may have a compact continuous matrix, a porous structure, a network filled with liquids, or can be an interpenetrating network, or may exhibit a capsule-type structure with a core and one or more envelope layers, or may be composed of a large number of single particles. For the purposes of the invention it is important that the particles are capable of undergoing a directed change in shape from a non-spherical shape into a spherical shape, which is induced by a suitable stimulus. The particle preferably consists of a porous material/scaffold or of a large number of smaller particles.

The term “stimulus” in the meaning of the present application is understood to be any exposure of the particles that causes the particle to return to its permanent spherical shape. For example, the stimulus can be a change in temperature or a change in chemical environment, such as pH value or the like. It is also conceivable that recovery is triggered by degradation of structures of the particle, by means of which the particle has been fixed in its non-spherical shape.

The term “uptake” describes the incorporation of particles in active cells, particularly in cells capable of presenting antigens, such as macrophages, dendritic cells, Langerhans cells, B lymphocytes, etc. Uptake may proceed via all biologically possible mechanisms, including receptor-mediated and non-receptor-mediated mechanisms. In one embodiment, cellular uptake proceeds via phagocytosis, i.e. the particle is preferably prepared for uptake in cells by phagocytosis.

The term “shape memory material” as used herein relates to a material which can be transformed by means of suitable shaping processes from a given permanent shape into a temporary shape and fixed as such. When applying an external stimulus, the material will return to the original, permanent shape. The process of deforming and fixing the temporary shape is referred to as programming. The transition from the temporary shape into the permanent shape is subsumed under the term “recovery”. Similarly, the term “shape memory effect” used herein is understood in the sense as above and is not restricted to the use of shape memory materials, but generally describes a shape change induced by a stimulus.

The particle with inducible change of shape consists of materials which can be biostable or completely or partially biodegradable. In a preferred embodiment the particle completely or partially consists of a shape memory material, especially a shape memory polymer, shape memory hydrogel, or stimulus-sensitive gel. The shape memory material is preferably a shape memory polymer which has switching segments and crosslinking sites and is frequently characterized by a low water content <5% (m/m) and a low water absorption capacity <5% (m/m). In another preferred embodiment the particle completely or partially consists of a shape memory gel with a high water content >5%. In yet another preferred embodiment the particle completely or partially consists of a stimulus-sensitive gel. In another preferred embodiment the particle is a capsule with a core and an envelope which in turn can be constituted of several layers.

In a preferred variant the capsule core consists of a swellable gel, a stimulus-sensitive gel or a shape memory gel. The capsule has a permanent spherical shape which can be transformed into the temporary, non-spherical shape. Trans-formation of the capsule into the non-spherical shape and fixing in this shape are performed with the aid of the capsule envelope. In a preferred variant the capsule envelope completely or partially consists of a shape memory polymer. In another preferred variant the capsule envelope consists of a waxy substance. As a result of capsule envelope shaping, the core, which has a spherical basic shape, is elastically deformed and forced into the temporary, non-spherical and thus non-uptakeable or only slightly uptakeable shape.

The particles in their spherical shape preferably have a size allowing phagocytosis. The mean particle diameter in the uptakeable shape of the particles is preferably 0.1 to 100 μM, particularly 0.5 to 10 μm. The particles put to use have a shape allowing no or only limited uptake, and the particle in this temporary shape is preferably in the form of an oblate ellipsoid, a prolate ellipsoid, elliptical disc, rectangular disc, a so-called UFO shape (cf. Proc. Natl. Acad. Sci. 103 (2006) pp. 4930-4934), rod shape or worm shape. The temporary shape is transformed into a spherical, uptakeable shape by a suitable stimulus.

The particle can be used as carrier for biologically active substances. The term “biologically active substance” or “active substance” encompasses all pharmacologically active substances, including immunological material or diagnostic agents and mixtures thereof. The term also encompasses drugs, contrast agents or radioactive labels and the like. It also comprises precursors being converted into their active forms in the body, active and inactive conformations and isomers, and pharmacologically and/or immunologically active fragments thereof. More specifically, the active substance is a protein, peptide, glycoprotein, glycopeptide, glycolipid, lipoprotein, lipopeptide, cytokine, polysaccharide, DNA, RNA or an agonist or antagonist or inhibitor of receptors, enzymes or transporters of the active cells capable of particle uptake. Furthermore, the term encompasses vaccines of all types, including bacteria, viruses, cleavage products and synthetic analogs thereof, as well as immunomodulators and immunopotentiators. The term also encompasses cytostatic agents, immunosuppressants, glucocorticoids, toll-like receptor ligands, NOD-like receptor ligands, antiproliferative agents, enzyme inhibitors, statins. Furthermore, the term encompasses hydrophilic, hydrophobic and amphiphilic substances.

Active substance-loaded particles with inducible shape change can be used to achieve temporal and local switching of the biological effects caused by these active substances. The active substance is preferably embedded in the particle matrix, or the particle has a cavity wherein the active substance is incorporated. Embedding the active substance can also be effected by covalent binding to the matrix material, e.g. by means of a bond which can undergo intracellular hydrolytic, enzymatic or redox-chemical cleavage.

The use of vaccines in combination with the particles according to the invention provides the option of applying a mixture of immediately available vaccine and initially non-phagocytable and thus immunologically non-effective particles instead of the well-known multiple vaccination. It is only at a later stage that the particles are released by a specifically induced shape change for immunological recognition, phagocytosis and intracellular immunological processing.

It is preferred that the release rate of active substance from the particles in the temporary shape is lower than that in the spherical shape. In other words, the induced shape change provides e.g. easier access to the cavities so that diffusion of the active substance is enhanced.

In addition to the active substance optionally present, the particles used according to the invention therefore consist of, among other things, (i) shape memory polymers, (ii) shape memory gels, (iii) stimulus-sensitive hydrogels, or (iv) represent single- or multi-layered capsules with a core of a defined shape which is maintained in the programmed shape by one or more layers of the capsule envelope made of a different material.

The particles can be loaded with bioactive molecules either directly during shaping the permanent particle shape, using any known method of particle production (for example, emulsion, spraying, coacervation processes; Int. J. Pharm. 364 (2008) 298-327), or in subsequent operations (using e.g. sources in suitable solutions of the substances). The substances can be released in a diffusion- or degradation-controlled manner. In some embodiments the release of active substance can be initiated independently of the switched shape change, at the same time, or initiation can be triggered by the latter. Release of the particle load proceeds during a period of from seconds to years, preferably during 1 hour to 4 weeks.

The materials used in shaping a temporary particle shape can be both low-molecular weight substances and polymers, and in a preferred fashion, shape memory polymers, shape memory hydrogels, polymer-based stimulus-sensitive hydrogels, and, in the event of capsule envelopes, waxy substances are used. These materials are biocompatible with respect to the use in humans and animals and diagnostic uses performed in vivo, ex vivo and in vitro. The particles are constituted of the above-mentioned materials, optionally adjuvants <5% (m/m) and active substance (0.001%-80% [m/m]), and optionally one or more layers of a coating material. They can be i) biostable, i.e., the particle core will not be degraded in a biological medium during several years, ii) completely biodegradable to form water-soluble and excretable monomers to oligomers, iii) partially biodegradable, i.e., the entire particle matrix is degraded into fragments excretable and/or non-excretable from the body, or iv) excretable on routes other than chemical or biochemical degradation, e.g. decomposition of the particle constituted of physical aggregates into single particles or molecules of excretable size. Inter alia, the term “biodegradation” encompasses enzymatic mechanisms, hydrolytic mechanisms, as well as redox reactions such as cleavage of disulfide bridges.

As shape memory materials it is possible to use stimulus-sensitive shape memory polymers and shape memory gels having shape memory properties, the shape change of which is triggered by, for example, heat transfer (direct contact, or indirectly via electric, magnetic or other stimuli) under the influence of solvents and plasticizers, including water, by light or chemical or biochemical stimuli. This may involve physically or covalently crosslinked polymer networks.

Stimulus-sensitive gels can also be used as particle matrix. They have a high content of solvents of >5% (m/m), preferably in the range of from 20 to 99.999% (m/m). This involves a biocompatible solvent with respect to the field of use, such as dimethyl sulfoxide, N-methylpyrrolidone, but preferably water (the material is then referred to as hydrogel). As gelling agents, both small molecules (supramolecular gels, e.g. Chem. Soc. Rev. 2008, 37, 2699-2715) and polymer networks can be used. According to the present state of science, they can change their dimensions in response to e.g. changes in pH value, temperature, ionic strength, electric or magnetic fields, and in an enzyme-controlled manner. Apart from fixation of the particles in a non-spherical shape and switchable trans-formation into a spherical shape with the aid of said shape memory polymer, shape memory gel, stimulus-sensitive gel, it is also possible to use capsules with a particle core of swellable gels, shape memory gels or stimulus-sensitive gels. The particle core has a predetermined spherical shape which is assumed without external force. In the event of these particles, transformation into a non-uptakeable, i.e. non-spherical, particle shape is effected by means of a coating of a different material. Said different material can be a shape memory material. Alternatively, said different material can be a material having no shape memory effect, e.g. a waxy substance. This coating is applied prior to transformation into a non-spherical particle shape. The coating is capable of withstanding the restoring forces of the particle core so as to ensure preservation of the thus-programmed shape. The mechanical stability of the coating is lost upon exposure to suitable stimuli, e.g. thermal, electromagnetic, chemical or biochemical stimuli, gradual degradation in the body, or thermal transitions such as melting upon temperature rise. Shape recovery of the particle into the original shape, i.e. the spherical shape, takes place when transforming the coating into the spherical shape by applying stimuli, or if the restoring forces of the particle core predominate during degradation of the coating.

If a shape memory material is used to prepare the capsule envelope, a deformable material for the core will be sufficient. Recovery is then initiated by exposing the capsule envelope to suitable stimuli such as thermal, electromagnetic, chemical or biochemical stimuli.

Programming of the particles is effected on single particles or, in a convenient setup, by simultaneously programming a large number of particles, e.g. between plates optionally having a special surface texture and structure, or e.g. by means of a method using films. In the film method, programming of spherical into non-spherical particles is performed in such a way that the particles are incorporated in a polymer matrix such as a polymer film, e.g. a water-soluble polymer film, stretched under suitable conditions (e.g. elevated temperature) in one, two, or three dimensions, or fixed, and the non-spherical particles are subsequently isolated by dissolving the film. The above-mentioned process has been described in the literature in the context with irreversible deformation of polystyrene particles (Colloid Polym. Sci. 271 (1993) pp. 469-479; Proc. Natl. Acad. Sci. 104 (2007) pp. 11901-11904).

When administered into the tissue (e.g. intramuscularly, subcutaneously or intradermally), particles with diameters smaller than 10 μm, especially smaller than 5 μm, can be taken up by macrophages or dendritic cells and removed via the lymphatic organs. Special cells, so-called foreign body giant cells, can also take up larger particles. As shown in vitro, macrophages phagocytize non-spherical microparticles to a lesser extent. Following injection, the non-spherical particles according to the invention with reduced cellular uptake can be transformed into a spherical shape by an externally stimulated change in shape, taken up in active cells, processed inside the cells, transported into the lymphatic organs, and a load possibly present can be recognized immunologically. For example, this can be utilized in a new principle of multiple vaccinations in humans and animals with only a single administration, wherein mixtures of an immediately available vaccine (dissolved or in spherical particles) and non-spherical particles are used and, instead of an additional administration, particles are fractionally converted into an immunologically recognizable form. Furthermore, this principle can be utilized in diagnostic issues, e.g. clarification of changes in the phagocytic activity of cells caused physiologically, pathologically or by immunosenescence.

The invention will be explained in more detail below in an example and with reference to the drawings wherein:

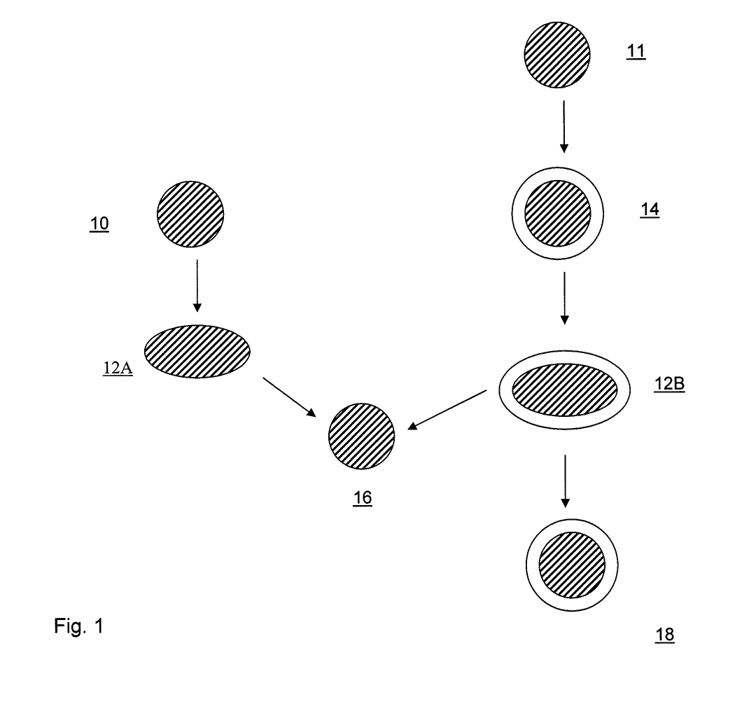

FIG. 1 shows a schematic representation of two ways in which the inventive particle with shape memory effect can be designed;

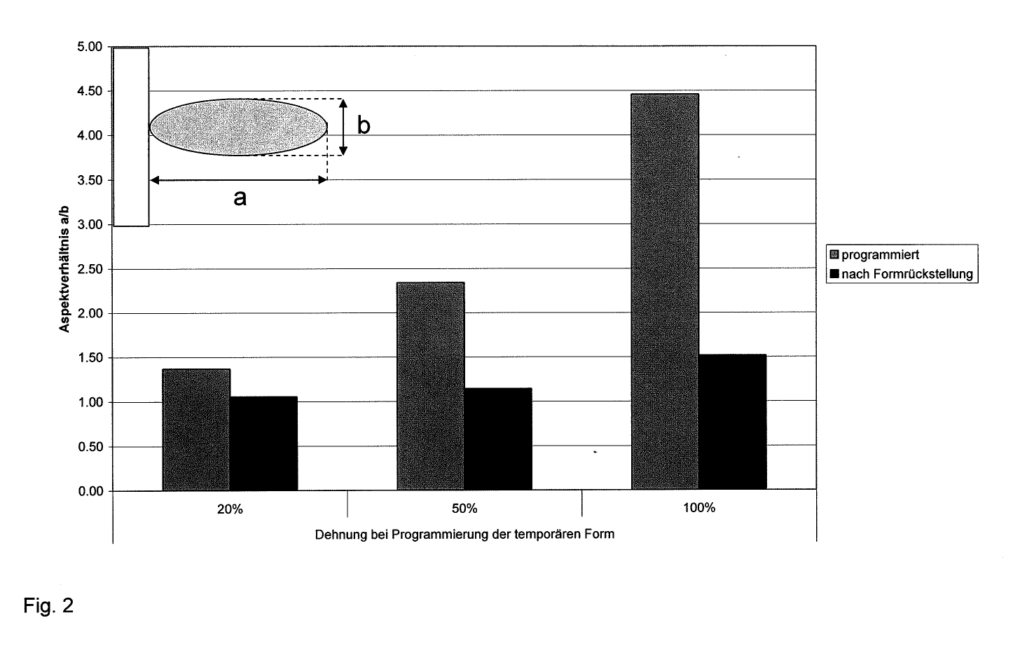

FIG. 2 shows the aspect ratios of microparticles in programmed shape and after shape recovery; and

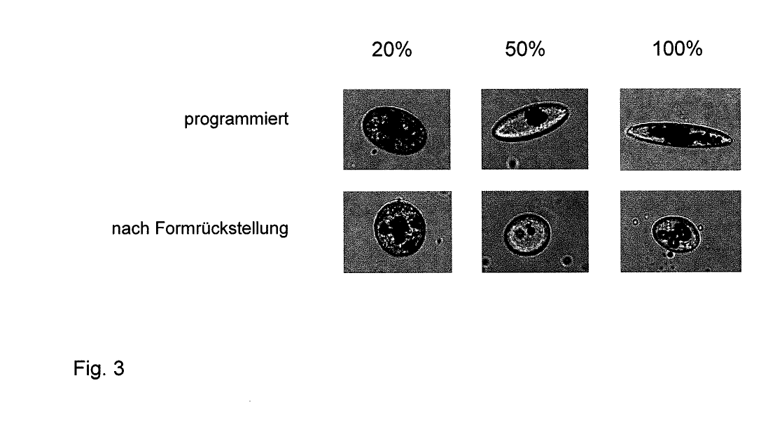

FIG. 3 shows microscopic images of microparticles of a shape memory polymer in temporary shape, programmed shape and after shape recovery into the permanent shape.

In a first step, spherical particles 10 with a shaping allowing cellular uptake are prepared from a material suitable for the desired application. In the embodiment shown on the left of FIG. 1 the material is a shape memory polymer, a shape memory gel or a stimulus-sensitive gel optionally loaded with an active substance. As a result of programming, the particle 10 is fixed in a temporary, non-spherical and thus non-phagocytable or only slightly phagocytable shape 12A. The particle is transformed into the spherical and thus phagocytable shape 16 by a suitable stimulus, e.g. a change in temperature, pH value, ionic strength, or exposure to light, and—following phagocytosis—can furnish the optionally desired biological effects.

According to a second variant, which is illustrated on the right of FIG. 1, the spherical particle 11 consists of a swellable gel, a shape memory gel or a stimulus-sensitive gel and can undergo elastic or plastic deformation. According to this variant, the particle 11 is initially coated with a suitable material, e.g. a wax or a shape memory polymer (coated particle 14), and subsequently fixed in a temporary, non-spherical and thus non-phagocytable or only slightly phagocytable shape 12B using a shaping procedure. The particle 12B is transformed into the spherical and thus phagocytable shape 16 by a suitable stimulus, in this case e.g. hydrolytic decomposition of the coating. Using other suitable stimuli with no decomposition of the coating, the particle undergoes transformation into the spherical and thus phagocytable shape 18 which can be similar to shape 14.

FIG. 2 shows the aspect ratios of microparticles in programmed shape and after shape recovery. Microscopic images of corresponding microparticles from a shape memory polymer can be inferred from FIG. 3. The particles are in their temporary, programmed shapes, with elongations of 20%, 50% and 100%. Following shape recovery into the permanent shape, spherical particles are present again.

1. A particle with inducible change of shape, said particle being suitable for cellular uptake, assuming a temporary, non-spherical and thus non-uptakeable or only slightly uptakeable shape prior to use, said shape being transformable into a spherical shape, i.e. uptakeable in cells, by a suitable stimulus.

2. The particle according to claim 1, said particle completely or partially consisting of a shape memory polymer, shape memory hydrogel or stimulus-sensitive gel.

3. The particle according to claim 1, said particle being a capsule with a deformable core made of a swellable gel, a shape memory gel or a stimulus-sensitive gel and a capsule envelope made of said shape memory polymer, which encloses the core.

4. The particle according to claim 1, which particle consists of a porous material/scaffold or of a multitude of smaller particles.

5. The particle according to claim 1, said particle being a capsule with a deformable core made of a swellable gel, a shape memory gel or a stimulus-sensitive gel and a capsule envelope made of a non-shape-memory material, which encloses the core, said core having a spherical basic shape which, as a result of capsule envelope shaping, is elastically deformed and forced into the temporary, non-spherical shape which therefore cannot be taken up in cells or only to a minor extent.

6. The particle according to claim 1, said particle in the shape uptakeable in cells having a mean particle diameter of from 0.1 to 100 μm.

7. The particle according to claim 6, said particle in the shape uptakeable in cells having a mean particle diameter of from 0.5 to 10 μm.

8. The particle according to claim 1, said particle in its temporary shape being in the form of an oblate ellipsoid, prolate ellipsoid, elliptical disc, rectangular disc, UFO shape, rod-shaped particle or worm-shaped particle.

9. The particle according to claim 1, said particle including one or more active substances.

10. The particle according to claim 9, said active substance being embedded or bound in the particle matrix or portions of the particle matrix.

11. The particle according to claim 9, the release rate of active substance from the particle in the temporary shape being lower than that in the phagocytable shape.

12. The particle according to claim 9, wherein said active ingredient includes a drug, a vaccine, proteins, peptides, glycoproteins, glycopeptides, lipoproteins, lipopeptides, glycolipids, cytokines, polysaccharides, DNA, RNA, glucocorticoids, immunosuppressants, immunomodulators, immunopotentiators, cytostatic agents, toll-like receptor ligands, NOD-like receptor ligands, antiproliferative agents, enzyme inhibitors, statins or surfactants.

13. The particle according to claim 1, said particle being prepared for uptake in cells by phagocytosis.

14. The particle according to claim 2, which particle consists of a porous material/scaffold or of a multitude of smaller particles.

15. The particle according to claim 3, which particle consists of a porous material/scaffold or of a multitude of smaller particles.

16. The particle according to claim 2, said particle in the shape uptakeable in cells having a mean particle diameter of from 0.1 to 100 μm.

17. The particle according to claim 3, said particle in the shape uptakeable in cells having a mean particle diameter of from 0.1 to 100 μm.

18. The particle according to claim 10, the release rate of active substance from the particle in the temporary shape being lower than that in the phagocytable shape.

19. The particle according to claim 10, wherein said active ingredient includes a drug, a vaccine, proteins, peptides, glycoproteins, glycopeptides, lipoproteins, lipopeptides, glycolipids, cytokines, polysaccharides, DNA, RNA, glucocorticoids, immunosuppressants, immunomodulators, immunopotentiators, cytostatic agents, toll-like receptor ligands, NOD-like receptor ligands, antiproliferative agents, enzyme inhibitors, statins or surfactants.

20. The particle according to claim 11, wherein said active ingredient includes a drug, a vaccine, proteins, peptides, glycoproteins, glycopeptides, lipoproteins, lipopeptides, glycolipids, cytokines, polysaccharides, DNA, RNA, glucocorticoids, immunosuppressants, immunomodulators, immunopotentiators, cytostatic agents, toll-like receptor ligands, NOD-like receptor ligands, antiproliferative agents, enzyme inhibitors, statins or surfactants.
